(19) United States
(12) Patent Application Publication
(10) Pub. No.: US 2006/0134184 A1
(43) Pub. Date: Jun. 22, 2006

(54) At least partially degradable films containing an active ingredient and method for the production of composition comprising an active compound

(75) Inventor: Gunter Helling, Odenthal (DE)

Correspondence Address: CONNOLLY BOVE LODGE & HUTZ, LLP, P O BOX 2207, WILMINGTON, DE, 19899, US

(73) Assignee: AgfaPhoto GmbH, Leverkusen (DE)

(21) Appl. No.: 10/515,802

(22) PCT Filed: Apr. 4, 2003
(86) PCT No.: PCT/EP03/50092

§ 371 Date: Nov. 28, 2005

(51) Int. Cl. A61K 9/28; A61K 9/70; B05D 3/02

(52) U.S. Cl. 424/443; 427/2.14

(57) ABSTRACT

A film which comprises at least one active compound and is at least partly degraded under the conditions of the site of action is suitable as a new presentation form of active compounds, in particular those having a pharmacological or biocidal action.

The invention relates to a composition which comprises at least one active compound and with which an accurate, controlled and regulatable release of the active compound of at least one is possible.

Active compounds in this context are understood as meaning low molecular weight organic compounds which, after their release from the composition, unfold a desired action, in particular a pharmacological or biocidal action.

Pharmaceutical active compounds (medicaments) are available in a large number of presentation forms which should make it possible for the pharmaceutical active compound to unfold its action at the desired site in an organism at the desired time and in the desired dosage. These aims could not always be fulfilled with the known presentation forms.

There is therefore a constant object of developing new improved presentation forms which render possible specific applications and dosages.

This object is achieved according to the invention by a film which comprises the active compound of at least one and is at least partly degraded under the conditions of the site of action.

The film is preferably made of biological material, for example an optionally partly degraded polypeptide, in particular gelatine. The biological material is preferably degraded enzymatically at the site of action and in this way releases the active compound of at least one stored in it.

The biological material of the film can be modified, for example by aqueous polymer latices or polymer dispersions, by water-soluble, optionally crosslinkable polymers or copolymers or protein derivatives. Examples of these are described in Res. Discl. 38 957 (1996) on p. 598, chapter II A and chapter II C, which comprises e.g. a polyacrylamide, a polyacrylic acid derivative, a polyvinylpyrrolidone, a polyvinyl alcohol, a polyvinylimidazole, a polyethylene oxide, a polystyrenesulfonic acid or a cellulose derivative. The biological material can furthermore be cured, that is to say crosslinked, to a varying extent, e.g. by a crosslinking via amino groups by means of bisvinyl sulfones, aldehydes, cyanuric chlorides or epoxides or by a crosslinking via carboxyl and amino groups by means of carbodiimides or carbamoylpyridium salts or by a crosslinking via carboxyl groups by means of aziridine derivatives. Further suitable curing agents are described in Res. Discl. 38 957 (1996) on p. 599, chapter II, B.

The films of biological material, in particular of gelatine, have many advantages:

They can be produced

as thin layers,

as exact layers,

with many layers,

as layers with predetermined properties,

as flexible layers.

They are produced, in particular, by coating the biological material on which the films are based as an aqueous solution on a carrier film by means of a suitable coating device and peeling it off after drying. This is advantageously carried out continuously with a cascade or curtain coater.

The coating devices are known, for example, from the production of photographic materials.

Possible carrier films are, in particular, cellulose triacetate film, and paper coated with polyethylene on both sides, both of which are known as carrier materials for photographic materials. Films of polyester (PET or PEN) or polycarbonate are furthermore also possible.

In contrast to photographic materials, the carrier films are not equipped with substrate or adhesive layers, which as is known are intended to bind the subsequent coated layers to the carrier film, since the layer coated is to be peeled off after the production.

The active compounds are added to the coating solutions and incorporated into the later films in this way. An exact dosage is possible here. Furthermore, the active compound can be introduced e.g. distributed in a specific environment, for example in a solution in a high-boiling organic solvent which is emulsified in the aqueous coating solution.

The active compound can also be introduced in the form of a loaded latex. In this case, a latex is impregnated or loaded with the water-soluble active compound. This method of introduction is described for photographic colour couplers in Res. Discl. 38 957 (1996), p. 623.

The film according to the invention can comprise several layers. This is of advantage if a film is to comprise at least two active compounds which are incompatible with one another, since they can be introduced into different layers. It is also possible to provide active compounds in various concentrations in the various layers, in order to render possible a controlled release of the active compound in a varying amount during later use.

To produce a pharmaceutical presentation form the film can be cut into small and very small parts and applied in this way, whether it is a solid or liquid presentation form contained in one of these parts, for example in a tablet or in a juice.

It is furthermore possible to use larger pieces of film in the form of a patch.

The release takes place by degradation of the film substance, and in the case of a gelatine film by enzymatic degradation, in particular by an enzyme of the organism which is to be treated with the active compound, for example with trypsin.

If a particularly slow administration of the active compound and associated with this a particularly slow degradation of the film is to be achieved, it is possible to provide the film with non-degradable protective layers on both sides, which can already be effected during the production, in order to render possible a degradation and hence a release of active compound only via the cut edges of the small pieces of film.

Further possibilities of feeding individual or several active compounds to an organism in the desired manner emerge from the above statements.

### EXAMPLES

Preparation of the Active Compound Emulsions

Emulsion 1

9 g metipranolol and 10.0 g olive oil are dissolved in 70.0 g ethyl acetate and the solution is emulsified into an aqueous solution of 10.0 g gelatine, 350 g water and 0.5 g dodecylbenzenesulfonate by means of an emulsifying apparatus at 40° C. in the course of 10 min. During this the ethyl acetate is evaporated and an O/W emulsion is obtained, which is topped up to 500 g with water.

Emulsion 2

12.0 g betaxolol and 90.0 g olive oil are dissolved in 80 g ethyl acetate and the solution is emulsified into an aqueous solution of 6.0 g gelatine, 350 g water and 0.5 g dodecylbenzenesulfonate by means of an emulsifying apparatus at 40° C. in the course of 12 min. During this the ethyl acetate is evaporated and an O/W emulsion is obtained, which is topped up to 500 g with water.

Preparation of the Active Compound Coating Solutions

Coating Solution 1

66 g 20 wt. % gelatine, 90 g water and 4.0 g 10 wt. % sulfosuccinic acid dioctyl ester, Na salt solution (wetting agent) are added to 160 g emulsion 1 at 40° C. and the mixture is filled up to 350 g with water.

Coating Solution 2

70 g 20 wt. % gelatine, 90 g water and 4.0 g 10 wt. % sulfosuccinic acid dioctyl ester, Na salt solution (wetting agent) are added to 160 g emulsion 2 at 40° C. and the mixture is filled up to 350 g with water.

Curing Agent Solution

9.2 g bisvinylsulfonylmethane are dissolved in 450 g of a mixture of 30 g ethanol and 420 g water, and 6 g of a 10 wt. % wetting agent solution based on a triterpene compound (saponin) are added.

Enzymatically Degradable Film

Using a cascade coater known from the production of photographic materials, a non-substrated polyethylene terephthalate film is coated with

1. coating solution 1

2. coating solution 2

3. coating solution 1

4. curing agent solution

The coating amount is chosen here such that after drying at 50° C. a composite of 3 layers with coating amounts of

layer 1 10 g/m²

Layer 2 20 g/m²

layer 3 10 g/m²

is obtained. The non-adhesive layer composite is peeled off from the polyethylene terephthalate film and cut into pieces 1 mm in size.

Test for Selective Release of Active Compounds

1. 0.5 g of the abovementioned pieces of film is dispersed in 100 g water buffered at pH 8 and the dispersion is stirred for 1 hour at 20° C. It is then filtered and the filtrate is analysed. No active compound is found.
2. 0.5 g of the abovementioned pieces of film is dispersed in 100 g water buffered at pH 8, 10 g of a 3 wt. % trypsin solution are added and the dispersion is stirred at 20° C. Samples are taken after 10 min (sample 1) and after 60 min (sample 2) and filtered and the filtrates analysed.

Result:
Sample 1 contains exclusively metipranolol
Sample 2 contains metipranolol and betaxolol

The test shows that the pieces of film release the active compounds selectively in succession by enzymatic degradation.

1. Process for the production of a film which comprises at least one active compound and is at least partly degraded under the conditions of the site of action, the film comprising various layers which comprise at least one active compound in various concentrations, characterized in that the coating is carried out with a cascade or curtain coater.

2. Process according to claim 1, characterized in that the active compound is a medicament.

3. Process according to claim 1, characterized in that the active compound is an active compound having a biocide action.

4. Process according to claim 1, characterized in that the film is made of an enzymatically degradable biological material.

5. Process according to claim 1, characterized in that the film is made of a polypeptide or a partly degraded polypeptide.

6. Process according to claim 1, characterized in that the film is made of gelatine.

7. Process according to claim 4, characterized in that the material forming the film is cured by application of a curing agent solution.

8. Process according to claim 1, characterized in that the film comprises various layers which comprise various active compounds.

9. Process according to claim 1, characterized in that the layers forming the film are coated as an aqueous solution on to a carrier film, dried and peeled off from the carrier film.

10. Process according to claim 9, characterized in that the carrier film is not equipped with substrate or adhesive layers.

11. Process according to claim 1, characterized in that the film is provided with non-degradable protective layers on both sides.

12. Process according to claim 1, characterized in that the active compound is introduced in the form of a loaded latex.

13. Process according to claim 1, characterized in that the active compound of at least one is introduced in the form of an oil-in-water emulsion.

14. Film comprising an active compound, produced by the process of claim 1.

15. Film comprising an active compound, produced by the process of claim 7.

16. Film comprising an active compound according to claim 14, characterized in that at least one of the layers comprises a wetting agent.

17. Film which comprises an active compound and is at least partly degraded under the conditions of the site of action, the film comprising various layers which comprise at least one active compound in various concentrations, characterized in that the material forming the film is cured by application of a curing agent solution and/or the film is provided with non-degradable protective layers on both sides and/or the active compound is introduced in the form of a loaded latex and/or the film comprises a wetting agent in at least one of the layers.

18. (canceled)

19. (canceled)

20. (canceled)

21. A process to feed individual or several active compounds to an organism in the desired manner which comprises using the film as claimed in claim 14.

22. A medicament or biocide which comprises the film as claimed in claim 14.

23. A patch which comprises the film as claimed in claim 14.
